(19) United States
(12) Patent Application Publication
(10) Pub. No.: US 2026/0215763 A1
(43) Pub. Date: Jul. 30, 2026

(54) METHOD FOR GENERATING MEDICAL IMAGE, MEDICAL APPARATUS, AND NON-TRANSITORY COMPUTER-READABLE MEDIUM

(71) Applicant: GE Precision Healthcare LLC, Waukesha, WI (US)

(72) Inventors: Gang Liu, Wuxi (CN); Longfei Wu, Wuxi (CN); Yuanlong Meng, Wuxi (CN); Min Zhou, Guangzhou (CN)

(21) Appl. No.: 19/446,707

(22) Filed: Jan. 12, 2026

(30) Foreign Application Priority Data
Jan. 10, 2025 (CN) 202510041160.9

(51) Int. Cl. A61B 8/00; A61B 8/08; G06T 7/73; G06T 15/08; G06V 20/50

(52) U.S. Cl. CPC A61B 8/5246; A61B 8/0875; A61B 8/481; A61B 8/483; A61B 8/5223; G06T 7/73; G06T 15/08; G06V 20/50; G06T 2200/04; G06T 2207/10136; G06T 2207/30012; G06T 2210/41; G06V 2201/033

(57) ABSTRACT

A method for generating a medical image including identifying a target object from a two-dimensional (2D) medical image to obtain information of the target object; setting a target position in the 2D medical image according to the information of the target object; extracting information corresponding to the target position from a three-dimensional (3D) medical image associated with the 2D medical image; and generating a target medical image based on the extracted information. A target position is automatically set based on an identification result of a target object, information in a 3D medical image is extracted based on the target position, and then a target medical image is generated

### CROSS-REFERENCE TO RELATED APPLICATION

This application claim priority to Japanese Patent Application No. 202510041160.9, which was file on Jan. 10, 2025 at the Chinese Patent Office. The entire contents of the above-listed application are incorporated by reference herein in their entirety.

### TECHNICAL FIELD

Embodiments of the present application relate to the technical field of medical imaging, and in particular, to a method for generating a medical image, a medical apparatus, and a non-transitory computer-readable medium.

### BACKGROUND

Medical imaging devices can non-invasively obtain internal tissue images of an object to be imaged. For example, a scanning device of the medical imaging device may scan a predetermined site of the object to be imaged to obtain imaging data containing information about the predetermined site.

Common medical imaging devices are, for example, ultrasound imaging systems, magnetic resonance imaging (MRI) systems, computed tomography (CT) systems, etc.

After a medical imaging device scans an object to be imaged, a two-dimensional (2D) medical image or a three-dimensional (3D) medical image is generated. A physician may make a diagnosis based on the 2D medical image or the 3D medical image.

It should be noted that the above introduction of the background is only for the convenience of clearly and completely describing the technical solutions of the present application, and for the convenience of understanding for those skilled in the art.

### SUMMARY OF THE INVENTION

In some cases, when viewing a 3D medical image, a physician sometimes needs to specify a local region of the 3D medical image, and a medical imaging device generates a target medical image that is convenient to observe based on data of the local region.

The inventors of the present application have found that when specifying a local region of a 3D medical image, a physician typically manually draws a line or a border on the image using an input device such as a mouse or a touch screen to specify the position of the local region, but such a method has some limitations: for example, when the physician manually specifies the position of the local region, inaccurate positioning may occur; and for another example, manually specifying the position of the local region increases the workload of the physician and reduces the efficiency of target medical image generation.

In order to resolve at least one technical problem described above or similar technical problems, embodiments of the present application provide a method for generating a medical image, a medical apparatus, and a non-transitory computer-readable medium. In the method for generating a medical image, a target position is automatically set based on an identification result of a target object, information in a 3D medical image is extracted based on the target position, and then a target medical image is generated. In this way, physicians are not required to manually set target regions. The method not only improves the accuracy of target position setting, but also reduces the workload of physicians, thereby enhancing the efficiency of target medical image generation.

According to an aspect of the embodiments of the present application, a method for generating a medical image is provided. The method comprises:
identifying a target object from a 2D medical image to obtain information of the target object;
setting a target position in the 2D medical image according to the information of the target object;
extracting information corresponding to the target position from a 3D medical image associated with the 2D medical image; and
generating a target medical image based on the extracted information.

According to another aspect of the embodiments of the present application, a medical apparatus is provided. The medical apparatus comprises:
a data collection unit that collects data for a 2D medical image and a 3D medical image; and
a processing unit that executes the method for generating a medical image described above.

According to yet another aspect of the embodiments of the present application, a non-transitory computer-readable medium is provided. The non-transitory computer-readable medium stores a computer program, and when executed by a computer, the computer program causes the computer to execute the steps of the method as described in the above embodiments.

One of the beneficial effects of the embodiments of the present application is that: in the method for generating a medical image, a target position is automatically set based on an identification result of a target object, information in a 3D medical image is extracted based on the target position, and then a target medical image is generated. In this way, physicians are not required to manually set target regions. The method not only improves the accuracy of target position setting, but also reduces the workload of physicians, thereby enhancing the efficiency of target medical image generation.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a method for generating a medical image according to some embodiments of the present application;

FIG. 2 is a schematic diagram of a relationship between a two-dimensional medical image and a three-dimensional medical image;

FIG. 3 shows schematic diagrams of a 2D medical image and information of a target object in Operation 101;

FIG. 4 is a schematic diagram of a target position in Embodiment 1;

FIG. 5 is a schematic diagram of translating a region to obtain a 3D region;

FIG. 6 is a schematic diagram of a volume contrast imaging (VCI) image;

FIG. 7 is a schematic diagram of a target position in Embodiment 2;

FIG. 8 is a schematic diagram of translating a curve to obtain a curved surface;

FIG. 9 is a schematic diagram of a target medical image generated based on image information on the curved surface;

FIG. 10 is a schematic diagram of a target position in Embodiment 3;

FIG. 11 is a schematic diagram of translating a line segment to obtain a section;

FIG. 12 is a schematic diagram of a target medical image 1200 generated based on image information on the section;

FIG. 13 is a schematic diagram of a medical apparatus according to an embodiment of the present application; and

FIG. 14 is a schematic diagram of an ultrasound imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements from one another by title, but do not represent the spatial arrangement, temporal order, etc., of the elements, and the elements should not be limited by these terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies. The terms “pixel” and “voxel” may be used interchangeably.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise explicitly specified in the context. In addition, the term “according to” should be construed as “at least in part according to . . . ”, and the term “based on” should be construed as “at least in part based on . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

Some embodiments of the present application provide a method for generating a medical image.

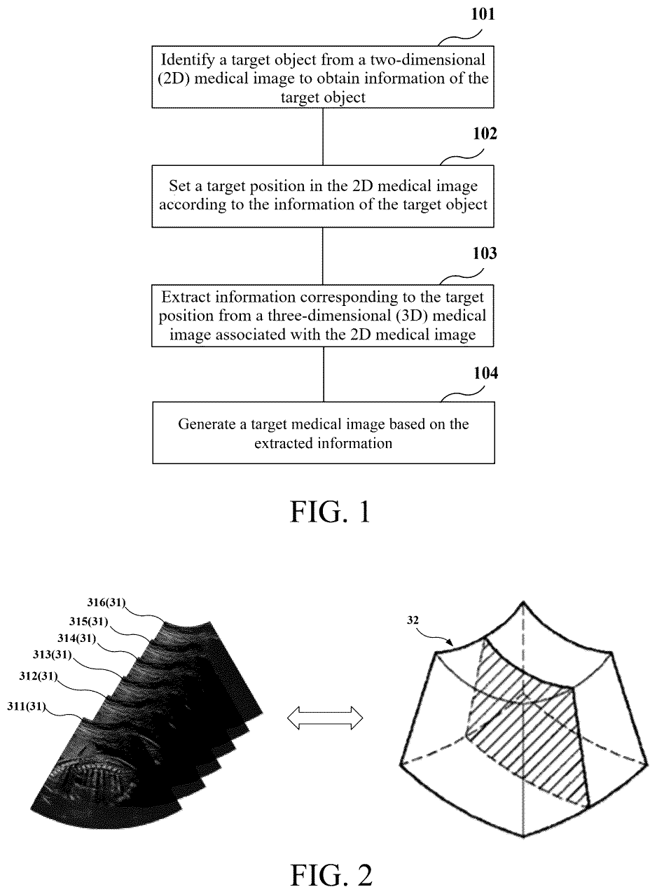

FIG. 1 is a schematic diagram of the method for generating a medical image according to some embodiments of the present application. As shown in FIG. 1, the method for generating a medical image includes:
Operation 101: identifying a target object from a two-dimensional (2D) medical image to obtain information of the target object;
Operation 102: setting a target position in the 2D medical image according to the information of the target object;
Operation 103: extracting information corresponding to the target position from a three-dimensional (3D) medical image associated with the 2D medical image; and
Operation 104: generating a target medical image based on the extracted information.

According to the embodiments of the present application, a target position is automatically set based on an identification result of a target object in a 2D medical image, information in a 3D medical image is extracted based on the target position, and then a target medical image is generated. In this way, physicians are not required to manually set target regions. The method not only improves the accuracy of target position setting, but also reduces the workload of physicians, thereby enhancing the efficiency of target medical image generation.

Additionally, in the present application, when the target position is automatically set in Operation 102, the target position may be adjusted by the physician (e.g., the target position is manually adjusted using an input device such as a mouse or a touch screen), thereby improving the flexibility of target position setting.

In the present application, the method for generating a medical image shown in FIG. 1 can be applied to a medical examination device, which may be a medical imaging device, such as an ultrasound imaging device, a magnetic resonance imaging (MRI) device, or a computed tomography (CT) imaging device. In addition, the medical examination device may alternatively be of another type, for example, an electrocardiogram examination device.

The 2D medical image in Operation 101 and the 3D medical image in Operation 103 may be an ultrasound medical image, a magnetic resonance medical image, or a CT medical image. In the following description of the present application, an ultrasound medical image is used as an example for description, and the description is also applicable to other types of medical images.

In some embodiments of the present application, the 2D medical image in Operation 101 is, for example, a 2D ultrasound medical image, and the 3D medical image in Operation 103 is, for example, a 3D ultrasound medical image.

In the present application, the 3D medical image may be generated based on data acquired by a mechanical scanning ultrasound probe, or the 3D medical image may be generated based on data acquired by an electronic scanning ultrasound probe.

For example, when the mechanical scanning ultrasound probe scans an object under examination, data (i.e., 2D data) on a plurality of scan planes is acquired by means of mechanical movement (e.g., oscillation) of internal components of the ultrasound probe, and the data on the plurality of scan planes is synthesized to generate a 3D medical image (e.g., a 3D ultrasound medical image).

For another example, the electronic scanning ultrasound probe may have detection elements arranged in an array, and when an object under examination is scanned, data (i.e., 2D data) on a plurality of scan planes are acquired by controlling detection directions of the detection elements, and a 3D medical image (e.g., a 3D ultrasound medical image) is generated based on the data on the plurality of scan planes.

In the present application, the 2D medical image in Operation 101 and the 3D medical image in Operation 103 may be associated.

In some examples, the 2D medical image is a portion of the 3D medical image.

FIG. 2 is a schematic diagram of a relationship between a two-dimensional medical image and a three-dimensional medical image.

As shown in FIG. 2, in an example, a plurality of 2D medical images 31 (e.g., a plurality of 2D medical images 311, 312, 313, 314, 315, 316) are synthesized to form a 3D medical image 32, and the plurality of 2D medical images 31 may be generated from data acquired by a mechanical scanning ultrasound probe. As shown in FIG. 2, in another example, a 3D medical image 32 may be segmented into a plurality of 2D medical images 31 (e.g., a plurality of 2D medical images 311, 312, 313, 314, 315, 316), and the 3D medical image 32 may be generated from data acquired by an electronic scanning ultrasound probe.

The 2D medical image in Operation 101 is one of the plurality of 2D medical images 31, e.g., the 2D medical image 313, and the 2D medical image 313 may be a standard image among the plurality of 2D medical images 31. The standard image may be a 2D medical image displaying a target object, wherein the target object includes, for example, at least one of a conus medullaris (cm) and a vertebral body (vb).

In some other examples, the 3D medical image may be generated based on the 2D medical image. For example, when an ultrasound probe (e.g., a mechanical scanning ultrasound probe or an electronic scanning ultrasound probe) of an ultrasound medical device is placed at a predetermined site of an object under examination in a certain posture, data acquired by the ultrasound probe can generate a 2D medical image (i.e., the 2D medical image in Operation 101) as a standard image, and then, using the posture and the placement site of the ultrasound probe as a reference, ultrasound scanning is performed on the object under examination and data is acquired, and a 3D medical image (i.e., the 3D medical image in Operation 103) is generated according to the acquired data. The standard image may be a 2D medical image displaying a target object, wherein the target object includes, for example, at least one of a conus medullaris (cm) and a vertebral body (vb).

In Operation 101 of the present application, the target object may be identified from the 2D medical image using a predetermined algorithm (e.g., the target object is identified by segmenting the target object from other objects in the image), thereby determining information of the identified target object in the 2D medical image. The predetermined algorithm may be an algorithm based on an artificial intelligence (AI) model. For example, the model may be a convolutional neural network (CNN) model or the like. The predetermined algorithm may further be an algorithm based on deep learning, an algorithm based on machine learning, or another algorithm. In another embodiment, the predetermined algorithm may also be a classical algorithm based on a non-AI model, for example, the target object may be identified by analyzing grayscale values of pixels in the medical image and comparing the grayscale values with a threshold to determine boundary information and the like in the image. The present application does not make a limitation.

In Operation 101 of the present application, the target object includes, for example, at least one of a conus medullaris (cm) and a vertebral body (vb). The present application is not limited thereto, and the target object may also be other sites of the object under examination. In the following description of the present application, using the target object including at least one of the conus medullaris (cm) and the vertebral body (vb) as an example, the related description content is equally applicable to when the target object is another site.

In Operation 101 of the present application, the information of the target object includes at least one of the position of the target object and the size of the target object.

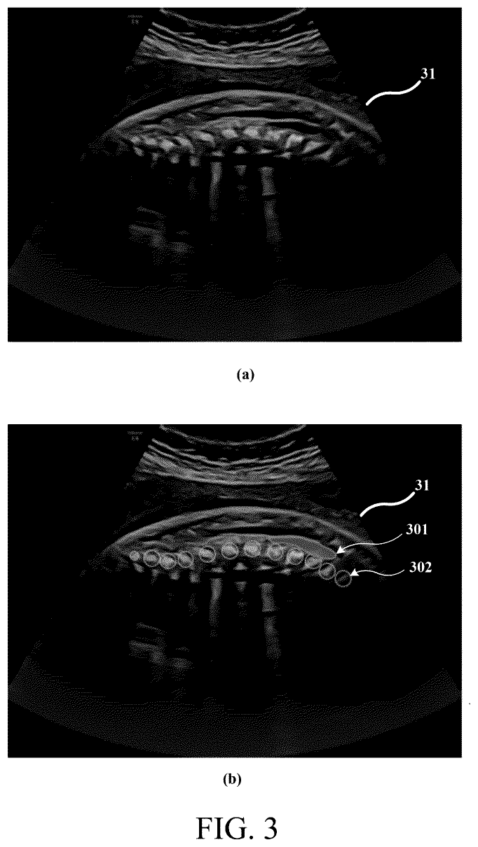

FIG. 3 shows schematic diagrams of the 2D medical image and the information of the target object in Operation 101. FIG. 3(a) shows the 2D medical image 31, for example, corresponding to the 2D medical image 313 in FIG. 2. The 2D medical image 31 is, for example, a 2D ultrasound medical image obtained by performing ultrasound scanning on the spine of a fetus. FIG. 3(b) shows the target object identified from the 2D medical image 31 in Operation 101, for example, the target object includes a conus medullaris (cm) 301 and a plurality of vertebral bodies (vb) 302.

For the example shown in FIG. 3, the information of the target object obtained in Operation 101 may be at least one of the following pieces of information:
a center position of the conus medullaris 301, for example, coordinates of a geometric center of the conus medullaris 301 in an image coordinate system of the 2D medical image 31;
the size of the conus medullaris 301, for example, the length (i.e., the size in an extension direction), the width, or the size in another direction of the conus medullaris 301;
a center position of each vertebral body 302, for example, coordinates of a geometric center of each vertebral body 302 in the image coordinate system of the 2D medical image 31; and
the size of each vertebral body 302, for example, a region occupied by each vertebral body 302 in the 2D medical image 31 is represented as an approximately circular shape, and the size of each vertebral body 302 is, for example, the diameter of a circular region occupied by each vertebral body 302, wherein the region of each vertebral body 302 is shown as a circle in FIG. 3(b).

In Operation 102 of the present application, the target position may be automatically set in the 2D medical image according to the information of the target object obtained in Operation 101. The target position may be represented by a line segment, a curve, or a region in the 2D medical image.

In Operation 103, the information corresponding to the target position in a 3D medical image is obtained based on the target position automatically set in the 2D medical image. For example, by extending, in the 3D medical image 32, the line segment, the curve, or the region representing the target position in the 2D medical image 31 in a direction perpendicular to the 2D medical image 31, a section, a curved surface, or a 3D region in the 3D medical image 32 can be obtained, and then image information in the section, the curved surface, or the 3D region is extracted.

The section, the curved surface, or the 3D region is merely an example, and the present application is not limited thereto. For example, in Operation 102, the target position may also be represented in other forms, and correspondingly, in Operation 103, the region corresponding to the target position in the 3D medical image may also have other shapes or positions. For example, the target position set in Operation 102 may be represented as an arc, a circle, or an ellipse, and in Operation 103, a spherical region or an ellipsoidal region in the 3D medical image may be obtained by performing a rotational operation (i.e., not limited to a translation operation such as extension) on the arc, the circle, or the ellipse in the 3D medical image. Then, image information within the spherical or ellipsoidal region, or image information on the surface of a sphere or an ellipsoid, can be extracted.

In the present application, since the 2D medical image is associated with the 3D medical image, in Operation 103, when the line segment, the curve, or the region representing the target position in the 2D medical image is subjected to a movement operation (e.g., a translation operation such as extension, or a rotational operation, etc.) in a predetermined direction in the 3D medical image, coordinate changes of each point on the line segment, the curve, or the region on a movement path can be determined, so that the coordinates of each point on the above-described section, curved surface, or 3D region can be determined in the 3D medical image, and then the image information within a corresponding range can be extracted based on the coordinates of each point.

In Operation 104, the target medical image is generated based on the information extracted in Operation 103. The target medical image can reflect the image information within the above-described section, curved surface, or 3D region, which assists physicians in observing the 3D medical image of the object under examination from a required angle, direction, or section, thereby improving the accuracy and efficiency of diagnosis based on the 3D medical image.

In the present application, the operations of Operation 102, Operation 103, and Operation 104 may also be different depending on different target medical images that need to be generated in Operation 104.

Hereinafter, Operation 102, Operation 103, and Operation 104 will be further described in conjunction with different embodiments. In the following embodiments, the target object identified from the 2D medical image 31 in Operation 101 is as shown in FIG. 3(b).

### Embodiment 1

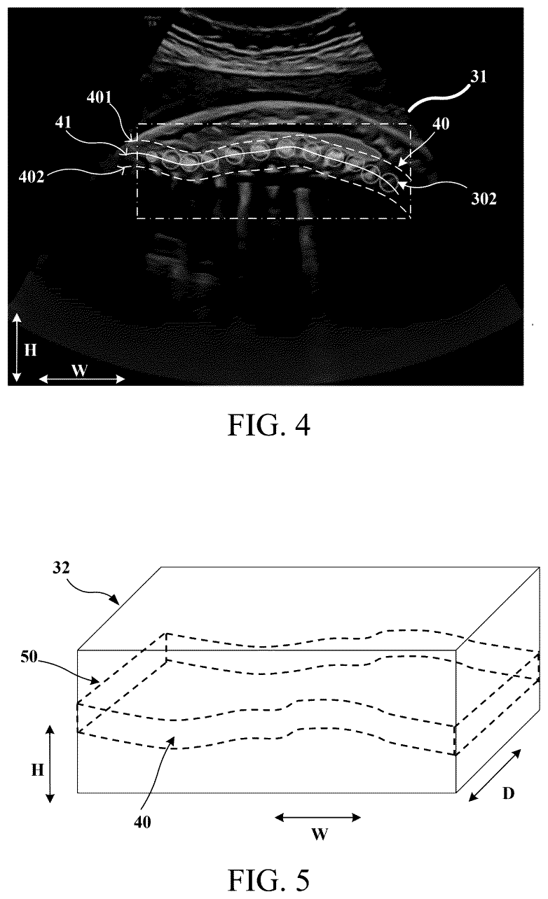

FIG. 4 is a schematic diagram of a target position in Embodiment 1.

As shown in FIG. 4, in Embodiment 1, according to information of a target object identified in Operation 101, a target position set in Operation 102 is represented as a region 40 having a predetermined height h with a line connecting the centers of a plurality of vertebral bodies 302 as a center line 41. A 2D medical image 31 has a height direction H and a width direction W, and the height direction H is perpendicular to the width direction W.

The predetermined height h of the region 40 refers to the size of the region 40 in the height direction H of the 2D medical image 31. For example, the region 40 has an upper boundary line 401 and a lower boundary line 402 in the height direction H, and the center line 41 is located between the upper boundary line 401 and the lower boundary line 402. For example, for any point on the center line 41, in the height direction H, the distance from the point to the upper boundary line 401 is equal to the distance from the point to the lower boundary line 402.

In some examples, the center line 41 may be formed by connecting the centers of the plurality of vertebral bodies 302 in the 2D medical image 31. In addition, the upper boundary line 401 may be obtained by translating the center line 41 upward in the height direction H, and the lower boundary line 402 may be obtained by translating the center line 41 downward in the height direction H.

The region 40 has the predetermined height h, that is, the distance between the upper boundary line 401 and the lower boundary line 402 in the height direction H is h. The predetermined height h may be set based on a maximum value of diameters of the plurality of vertebral bodies 302. For example, the diameters of the vertebral bodies 302 identified in the 2D medical image 31 are compared, a maximum diameter Dmax is selected, and h=k*Dmax is set, wherein k may be a coefficient not equal to 0, specifically, k may be greater than or equal to 1, or k may be other values.

Further, as shown in FIG. 4, the region 40 may have a predetermined width w, that is, the size of the region 40 in the width direction W of the 2D medical image 31 is w. In some examples, the predetermined width w may be set according to a distribution range of the plurality of vertebral bodies 302 identified from the 2D medical image 31. For example, if the distribution range of the plurality of vertebral bodies 302 in the width direction W of the 2D medical image 31 is w1, the predetermined width w is greater than w1, and thus, the plurality of vertebral bodies 302 (e.g., all of the vertebral bodies 302) are located within the region 40.

In Embodiment 1, information corresponding to the target position is extracted from a 3D medical image (e.g., the 3D medical image 32 in FIG. 2) via Operation 103. For example, the region 40 may be translated in the 3D medical image 32 such that the region 40 extends in a direction perpendicular to the region 40 to obtain a 3D region in the 3D medical image 32, and then image information within the 3D region is extracted (e.g., at least one type of image information such as the intensity, grayscale, and brightness of each pixel within the 3D region is extracted).

FIG. 5 is a schematic diagram of translating the region 40 to obtain a 3D region. As shown in FIG. 5, in the 3D medical image 32, the region 40 is translated in a direction perpendicular to the region 40 (i.e., direction D) to obtain a 3D region 50 in the 3D medical image 32.

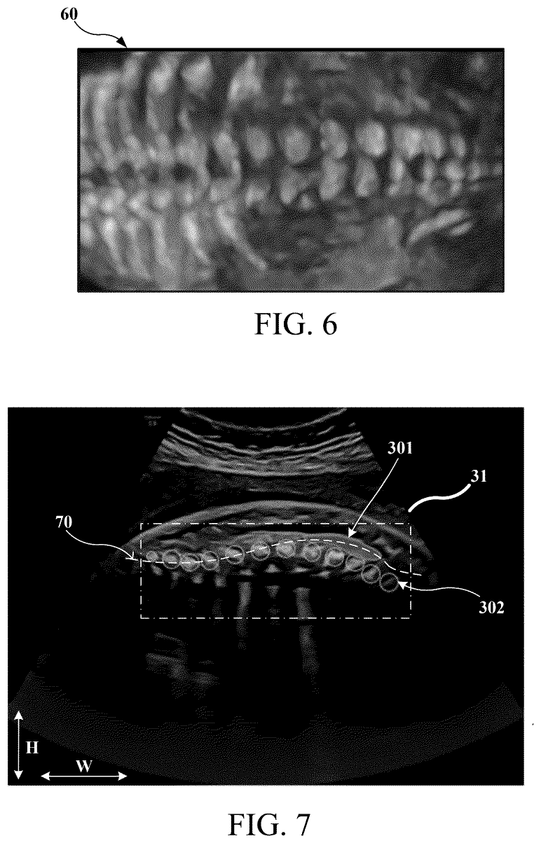

In Embodiment 1, via Operation 104, a target medical image is generated based on image information within the 3D region 50 extracted in Operation 103. In some examples, the target medical image may be a volume contrast imaging (VCI) image generated based on the image information within the 3D region 50. That is, in Operation 104, the image information within the 3D region 50 may be projected to a W-D plane (e.g., a max intensity projection is performed) in the height direction H to form a volume contrast imaging (VCI) image 60 shown in FIG. 6, the VCI image 60 being used as the target medical image.

In Embodiment 1 of the present application, the 3D region 50 can be intercepted near the height direction of the target object (e.g., the plurality of vertebral bodies 302) to generate the VCI image, which can prevent noise generated in the VCI image by interference information of other regions in the 3D medical image, so that the VCI image more accurately and clearly reflects the information of the target object, thereby facilitating improvement of the accuracy and efficiency of diagnosis. In contrast, if the 3D region is intercepted by manual setting to generate the VCI image, it is not only time-consuming and labor-intensive, but also a positioning deviation or a height (or thickness) deviation of the 3D region may occur, thereby introducing more noise into the VCI image and affecting the accuracy and efficiency of diagnosis.

### Embodiment 2

FIG. 7 is a schematic diagram of a target position in Embodiment 2.

As shown in FIG. 7, in Embodiment 2, according to information of a target object identified in Operation 101, a target position set in Operation 102 is represented as a curve 70 passing through the center of a conus medullaris 301 and the center of at least one vertebral body 302.

A 2D medical image 31 has a height direction H and a width direction W, and the height direction H is perpendicular to the width direction W.

In Embodiment 2, information corresponding to the target position is extracted from a 3D medical image (e.g., the 3D medical image 32 in FIG. 2) via Operation 103. For example, the curve 70 may be translated in the 3D medical image 32 such that the curve 70 extends in a direction perpendicular to the 2D medical image 31 to obtain a curved surface in the 3D medical image 32, and then image information on the curved surface is extracted (e.g., at least one type of image information such as the intensity, grayscale, and brightness of each pixel on the curved surface is extracted).

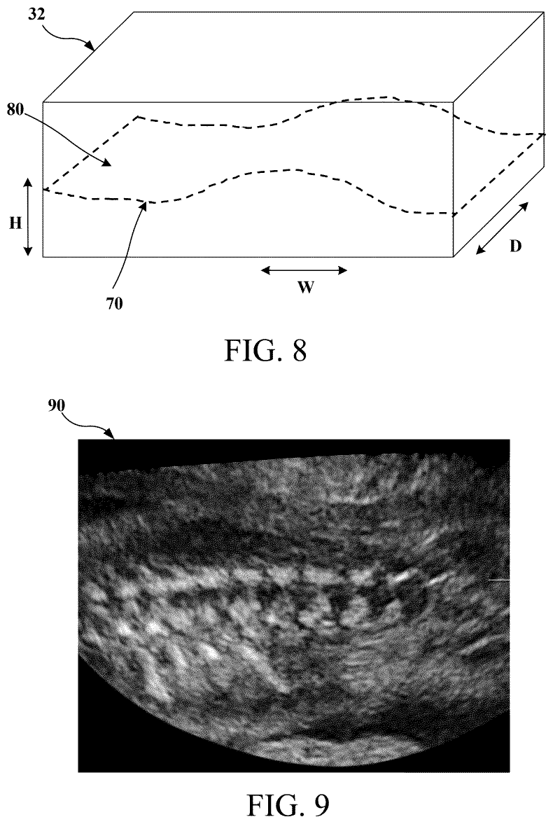

FIG. 8 is a schematic diagram of translating the curve 70 to obtain a curved surface. As shown in FIG. 8, in the 3D medical image 32, the curve 70 is translated in a direction (i.e., direction D) perpendicular to the height direction H and the width direction W to obtain a curved surface 80 in the 3D medical image 32.

In Embodiment 2, via Operation 104, a target medical image is generated based on image information on the curved surface 80 extracted in Operation 103 (e.g., the image information on the curved surface 80 is rendered).

FIG. 9 is a schematic diagram of a target medical image 90 generated based on the image information on the curved surface 80.

In Embodiment 2 of the present application, a curved surface of interest is intercepted for the target object (e.g., the conus medullaris 301 and at least one vertebral body 302) to generate the target medical image, enabling flexible and efficient acquisition of an image of a required observation cross section, thereby facilitating improvement of the accuracy and efficiency of diagnosis.

### Embodiment 3

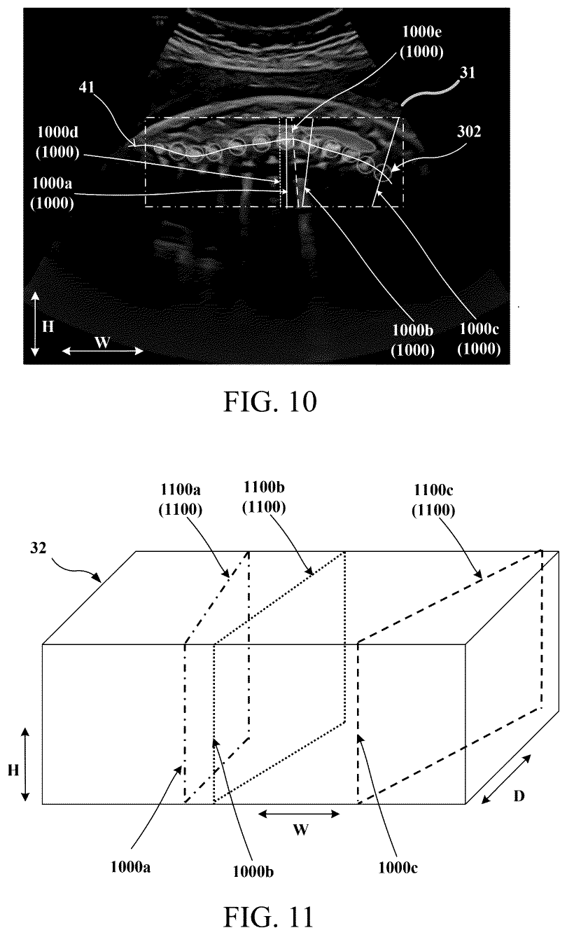

FIG. 10 is a schematic diagram of a target position in Embodiment 3.

As shown in FIG. 10, in Embodiment 3, according to information of a target object identified in Operation 101, a target position set in Operation 102 is represented as a line segment 1000 passing through at least one vertebral body 302. For example, the line segment 1000 may pass through the center of the vertebral body 302. Line segments at different positions may pass through different vertebral bodies 302. In FIG. 10, line segments 1000a, 1000b, 1000c, etc., are illustrated.

A 2D medical image 31 has a height direction H and a width direction W, and the height direction H is perpendicular to the width direction W.

In Embodiment 3, information corresponding to the target position is extracted from a 3D medical image (e.g., the 3D medical image 32 in FIG. 2) via Operation 103. For example, the line segment 1000 may be translated in the 3D medical image 32 such that the line segment 1000 extends in a direction perpendicular to the 2D medical image 31 to obtain a section in the 3D medical image 32, and then image information on the section is extracted (e.g., at least one type of image information such as the intensity, grayscale, and brightness of each pixel on the section is extracted).

FIG. 11 is a schematic diagram of translating the line segment 1000 to obtain a section. As shown in FIG. 11, in the 3D medical image 32, the line segment 1000 is translated in a direction (i.e., direction D) perpendicular to the height direction H and the width direction W to obtain a section 1100 in the 3D medical image 32. In FIG. 13, sections 1100a, 1100b, and 1100c correspond to the line segments 1000a, 1000b, and 1000c, respectively.

In Embodiment 3, via Operation 104, a target medical image is generated based on image information on the section 1100 extracted in Operation 103 (e.g., the image information on the section 1100 is rendered).

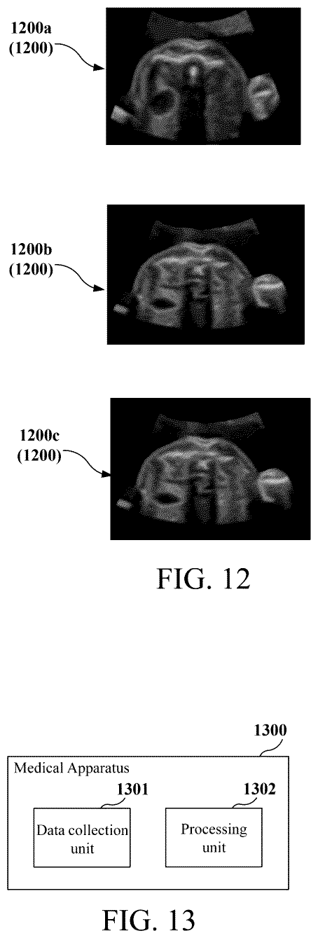

FIG. 12 is a schematic diagram of a target medical image 1200 generated based on the image information on the section 1100. In FIG. 12, target medical images 1200a, 1200b, and 1200c correspond to the sections 1100a, 1100b, and 1100c, respectively.

In Example 3, as shown in FIG. 10, at least one of the distance from each line segment 1000 to the center of the vertebral body 302 and the angle of each line segment 1000 relative to the vertebral body 302 is adjustable. For example, as shown in FIG. 10: the line segment 1000a passes through the center of the vertebral body 302 and is orthogonal to a center line 41 (regarding the definition of the center line 41, refer to Embodiment 1); the line segment 1000d does not pass through the center of the vertebral body 302, and the line segment 1000d is parallel to the line segment 1000a; and the line segment 1000e does not pass through the center of the vertebral body 302, and the line segment 1000e is not parallel to the line segment 1000a.

In some examples of Embodiment 3, corresponding parameters may be inputted by the physician to adjust the aforementioned distance and/or angle; alternatively, the distance and/or angle may be automatically adjusted based on a result of image identification or a result of image analysis so that optimal section information is presented to the physician.

In Embodiment 3 of the present application, a section of interest is intercepted for the target object (e.g., at least one vertebral body 302) to generate the target medical image, enabling flexible and efficient acquisition of an image of a section that needs to be observed, thereby facilitating improvement of the accuracy and efficiency of diagnosis.

Some embodiments of the present application further provide an apparatus for generating information.

FIG. 13 is a schematic diagram of a medical apparatus according to an embodiment of the present application.

As shown in FIG. 13, the medical apparatus 1300 includes:
a data collection unit 1301 that collects data for a 2D medical image and a 3D medical image; and
a processing unit 1302 that executes the method for generating a medical image as shown in FIG. 1.

In some examples, the target object includes at least one of a conus medullaris (cm) and a vertebral body (vb); and
the information of the target object includes at least one of the position and the size of the target object.

In some examples, the target position is represented as a region having a predetermined height with a line connecting the centers of a plurality of vertebral bodies as a center line.

In some examples, the predetermined height is set based on a maximum value of the diameters of the plurality of vertebral bodies.

In some examples, extracting information corresponding to the target position includes:
translating the region in a direction perpendicular to the region in the 3D medical image to obtain a 3D region in the 3D medical image; and
extracting image information within the 3D region.

In some examples, the target medical image is a volume contrast imaging (VCI) image generated based on the image information within the 3D region.

In some examples, the target position is represented as a curve passing through the center of the conus medullaris and the center of at least one vertebral body.

In some examples, extracting information corresponding to the target position includes:
translating the curve in a direction perpendicular to the 2D medical image in the 3D medical image to obtain a curved surface in the 3D medical image; and
extracting image information on the curved surface.

In some examples, the target position is represented as a line segment passing through at least one vertebral body.

In some examples, extracting information corresponding to the target position includes:
translating the line segment in a direction perpendicular to the 2D medical image in the 3D medical image to obtain at least one section in the 3D medical image; and
extracting image information on the section.

In some examples, at least one of the distance from the line segment to the center of the vertebral body and the angle of the line segment relative to the vertebral body is adjustable.

In some examples, the 2D medical image is a 2D ultrasound medical image, the 3D medical image is a 3D ultrasound medical image, and the 3D medical image is generated based on data acquired by an electronic scanning ultrasound probe or a mechanical scanning ultrasound probe.

For further description of each unit in the medical apparatus 1300, reference may be made to the detailed description of a corresponding operation in FIG. 1.

It can be understood that the medical apparatus 1300 may include different types, and for example, the medical apparatus may be a medical imaging system, such as an ultrasound imaging system, a magnetic resonance imaging (MRI) system, or a computed tomography (CT) imaging system. The following is described by taking an example in which the medical apparatus is an ultrasound imaging system.

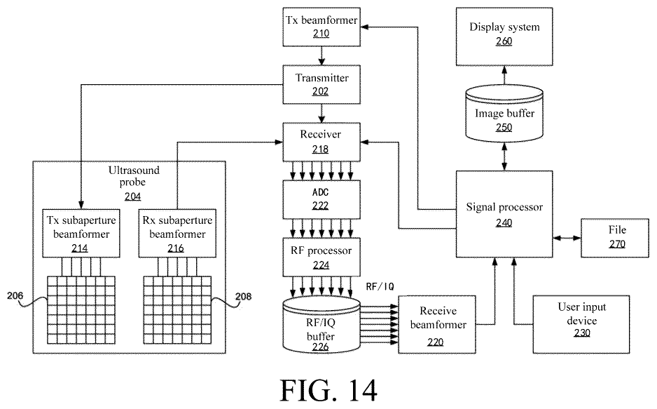

FIG. 14 is a schematic diagram of an ultrasound imaging system according to an embodiment of the present application. As shown in FIG. 14, the ultrasound imaging system 200 may be configured to provide ultrasound imaging, and thus may include suitable circuitry, interfaces, logic, and/or code for executing and/or supporting ultrasound imaging-related functions.

The ultrasound imaging system 200 includes, for example, a transmitter 202, an ultrasound probe 204 (corresponding to the foregoing scanning device 1), a transmit beamformer 210, a receiver 218, a receive beamformer 220, an RF processor 224, an RF/IQ buffer 226, a user input module 230, a signal processor 240 (corresponding to the foregoing processor 114), an image buffer 250, a display system 260 (a display), and a file 270.

The transmitter 202 may include suitable circuitry, interfaces, logic, and/or code operable to drive the ultrasound probe 204. The ultrasound probe 204 may include an array of 2D piezoelectric elements. The ultrasound probe 204 may include a set of transmit transducer elements 206 and a set of receive transducer elements 208 that typically form the same element. In some embodiments, the ultrasound probe 204 may be operable to acquire ultrasound image data covering at least a substantial portion of an anatomical structure (such as the heart or any suitable anatomical structure). The ultrasound probe 204 may be an electronic scanning ultrasound probe or a mechanical scanning ultrasound probe.

The transmit beamformer 210 may include suitable circuitry, interfaces, logic, and/or code that is operable to control the transmitter 202, and the transmitter 202 drives the set of transmit transducer elements 206 by means of a transmit subaperture beamformer 214 to transmit ultrasound emission signals into a region of interest (e.g., a person, animal, subsurface cavity, physical structure, etc.). The emitted ultrasound signal can be backscattered from structures in the object of interest (e.g., blood cells or tissue) to produce echoes. The echo is received by the receive transducer element 208.

The set of receive transducer elements 208 in the ultrasound probe 204 is operable to convert the received echo to an analog signal for subaperture beam formation by means of a receiving subaperture beamformer 216, which is then transmitted to the receiver 218. The receiver 218 may include suitable circuitry, interfaces, logic, and/or code that is operable to receive signals from the receiving subaperture beamformer 216. The analog signal can be transferred to one or more of a plurality of A/D converters 222.

The plurality of A/D converters 222 may include suitable circuitry, interfaces, logic, and/or code that is operable to convert the analog signal from the receiver 218 to a corresponding digital signal. The plurality of A/D converters 222 are provided between the receiver 218 and the RF processor 224. Nevertheless, the present application is not limited in this regard. Thus, in some embodiments, the plurality of A/D converters 222 may be integrated within the receiver 218.

The RF processor 224 may include suitable circuitry, interfaces, logic, and/or code that is operable to demodulate the digital signals outputted by the plurality of A/D converters 222. According to one embodiment, the RF processor 224 may include a complex demodulator (not shown) that is operable to demodulate the digital signal to form an I/Q data pair representing the corresponding echo signal. The RF or I/Q signal data can then be transferred to the RF/IQ buffer 226. The RF/IQ buffer 226 may include suitable circuitry, interfaces, logic, and/or code that is operable to provide temporary storage of RF or I/Q signal data generated by the RF processor 224.

The receive beamformer 220 may include suitable circuitry, interfaces, logic, and/or code that may be operable to execute digital beamforming processing to, for example, sum delay-channel signals received from the RF processor 224 via the RF/IQ buffer 226 and output a beam summing signal. The resulting processed information may be the beam summing signal outputted from the receive beamformer 220 and transmitted to the signal processor 240. According to some embodiments, the receiver 218, the plurality of A/D converters 222, the RF processor 224, and the beamformer 220 may be integrated into a single beamformer which may be digital. In various embodiments, the ultrasound imaging system 200 includes a plurality of receive beamformers 220.

The user input device 230 can be used to enter patient data, scan parameters, and settings, and select protocols and/or templates to interact with an artificial intelligence segmentation processor, so as to select tracking targets, etc. In an illustrative embodiment, the user input device 230 is operable to configure, manage, and/or control the operation of one or more components and/or modules in the ultrasound imaging system 200. In this regard, the user input device 230 is operable to configure, manage, and/or control the operation of the transmitter 202, the ultrasound probe 204, the transmit beamformer 210, the receiver 218, the receive beamformer 220, the RF processor 224, the RF/IQ buffer 226, the user input device 230, the signal processor 240, the image buffer 250, the display system 260, and/or the file 270.

For example, the user input device 230 may include a button, a rotary encoder, a touch screen, motion tracking, voice recognition, a mouse device, a keyboard, a trackball, a camera, and/or any other device capable of receiving user commands. In some embodiments, for example, one or more user input devices 230 may be integrated into other components (such as the display system 260 or the ultrasound probe 204). As an example, the user input device 230 may include a touch screen display. As another example, the user input device 230 may include an accelerometer, gyroscope, and/or magnetometer attached to and/or integrated with the probe 204 to provide pose and motion recognition of the probe 204, such as identifying one or more probe compressions against the patient's body, predefined probe movements, or tilt operations, etc. Additionally and/or alternatively, the user input device 230 may include image analysis processing to identify the probe pose by analyzing the acquired image data. The signal processor 240 may include suitable circuitry, interfaces, logic, and/or code that is operable to process the ultrasound scan data (i.e., the summed IQ signal) to generate an ultrasound image for presentation on the display system 260. The signal processor 240 is operable to execute one or more processing operations based on a plurality of selectable ultrasound modalities on the acquired ultrasound scan data. In an illustrative embodiment, the signal processor 240 is operable to execute display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various embodiments, the processed image data may be presented at the display system 260 and/or may be stored in the file 270. The file 270 can be a local file, a picture archiving and communication system (PACS), or any suitable device for storing images and related information.

The signal processor 240 may be one or more central processing units, microprocessors, microcontrollers, etc. For example, the signal processor 240 may be an integrated component, or may be distributed in various locations. The signal processor 240 may be configured to receive input information from the user input device 230 and/or file 270, generate outputs that may be shown by the display system 260, and manipulate the outputs, etc., in response to the input information from the user input device 230. The signal processor 240 may be capable of executing, for example, any of one or more of the methods and/or one or more sets of instructions discussed herein according to various embodiments.

In some embodiments of the ultrasound imaging system 200, the signal processor 240 can be configured to implement the functions of the data collection unit 1301 and the processing unit 1302 in FIG. 13, so as to execute the method for generating a medical image described in the foregoing embodiments of the present application. For example, the signal processor 240 receives data from the receive beamformer 220 and generates a 2D medical image and a 3D medical image based on the received data; further, the signal processor 240 identifies a target object from the 2D medical image to obtain information of the target object, a target position is set in the 2D medical image according to the information of the target object, information corresponding to the target position is extracted from the 3D medical image associated with the 2D medical image, and a target medical image is generated based on the extracted information. For a detailed description of the method for generating a medical image, reference may be made to the related descriptions of the foregoing embodiments.

The ultrasound imaging system 200 may be operated to continuously acquire ultrasound scan data at a frame rate suitable for the imaging situation under consideration. Typical frame rates are in the range of 20 to 220, but can be lower or higher. The acquired ultrasound scan data can be shown on the display system 260 in real-time at a display rate that is the same as the frame rate, or slower, or faster than the frame rate. The image buffer 250 is included to store processed frames of the acquired ultrasound scan data that are not scheduled for immediate display. Preferably, the image buffer 250 has sufficient capacity to store frames of ultrasound scan data for at least a few minutes. Frames of ultrasound scan data are stored in such a way that the frames of ultrasound scan data can be easily retrieved therefrom according to the acquisition sequence or time of the frames of ultrasound scan data. The image buffer 250 may be embodied in any known data storage medium.

In some specific embodiments, the signal processor 240 may be configured to execute or otherwise control at least some of the functions executed thereby based on user instructions via the user input device 230. As an example, a user may provide voice commands, probe poses, button presses, etc., to issue specific commands, such as controlling aspects of automatic strain measurement and strain ratio calculations, and/or providing or otherwise specifying various parameters or settings associated therewith, as described in more detail below.

In operation, the ultrasound imaging system 200 may be used to generate ultrasound images, including 2D, 3D, and/or four-dimensional (4D) images. In this regard, the ultrasound imaging system 200 may be operated to continuously acquire ultrasound scan data at a specific frame rate, which may be applicable to the imaging situation discussed. For example, the frame rate can be within the range of 20-70, or can be lower or higher. The acquired ultrasound scan data can be shown on the display system 260 at the same display rate as the frame rate, or slower or faster than the frame rate. The image buffer 250 is included to store processed frames of the acquired ultrasound scan data that are not scheduled for immediate display. Preferably, the image buffer 250 has sufficient capacity to store frames of ultrasound scan data for at least a few seconds. Frames of ultrasound scan data are stored in such a way that the frames of ultrasound scan data can be easily retrieved therefrom according to the acquisition sequence or time of the frames of ultrasound scan data. The image buffer 250 may be embodied in any known data storage medium.

In some cases, the ultrasound imaging system 200 may be configured to support grayscale and color-based operations. For example, the signal processor 240 may be operable to execute grayscale B-model processing and/or color processing. Grayscale B-model processing may include processing B-model RF signal data or IQ data pairs. For example, the grayscale B-model processing can enable the formation of an envelope of the beam-summed received signal by computing the amount (I²+Q²)^{1/2}. The envelope can be subjected to additional B-model processing, such as logarithmic compression, to form display data. The display data can be converted to X-Y format for video display. Scan-converted frames can be mapped to grayscale for display. The B model frame is provided to the image buffer 250 and/or the display system 260. Color processing may include processing color-based RF signal data or IQ data pairs to form frames to cover the B-model frames provided to image buffer 250 and/or display system 260. Grayscale and/or color processing may be self-adaptively adjusted based on user input (e.g., selections from the user input device 230), such as for enhancing the grayscale and/or color of a particular region.

The embodiments of the present application further provide a computer-readable program. The program, when executed, causes a computer to execute, in a medical imaging system, the method for generating a medical image as described in any of the foregoing embodiments.

The embodiments of the present application further provide a storage medium storing a computer-readable program. The computer-readable program causes a computer to execute, in a medical imaging system, the method for generating a medical image as described in any of the foregoing embodiments.

A non-transitory computer-readable medium stores a computer program. The computer program has at least one code segment, and the at least one code segment is executable by a machine (e.g., a computer) to cause the machine to execute the method for generating a medical image as described in any of the foregoing embodiments.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

What is claimed is:

1. A method for generating a medical image, comprising:
identifying a target object from a two-dimensional (2D) medical image to obtain information of the target object;
setting a target position in the 2D medical image according to the information of the target object;
extracting information corresponding to the target position from a three-dimensional (3D) medical image associated with the 2D medical image; and
generating a target medical image based on the extracted information.

2. The method according to claim 1, wherein
the target object comprises at least one of a conus medullaris and a vertebral body; and
the information of the target object comprises at least one of the position and the size of the target object.

3. The method according to claim 2, wherein
the target position is represented as a region having a predetermined height with a line connecting the centers of a plurality of vertebral bodies as a center line.

4. The method according to claim 3, wherein
the predetermined height is set based on a maximum value of the diameters of the plurality of vertebral bodies.

5. The method according to claim 3, wherein
extracting information corresponding to the target position comprises:
translating the region in a direction perpendicular to the region in the 3D medical image to obtain a 3D region in the 3D medical image; and
extracting image information within the 3D region.

6. The method according to claim 5, wherein
the target medical image is a volume contrast imaging (VCI) image generated based on the image information within the 3D region.

7. The method according to claim 2, wherein
the target position is represented as a curve passing through the center of the conus medullaris and the center of at least one vertebral body.

8. The method according to claim 7, wherein
extracting information corresponding to the target position comprises:
translating the curve in a direction perpendicular to the 2D medical image in the 3D medical image to obtain a curved surface in the 3D medical image; and
extracting image information on the curved surface.

9. The method according to claim 2, wherein
the target position is represented as a line segment passing through at least one vertebral body.

10. The method according to claim 9, wherein
extracting information corresponding to the target position comprises:
translating the line segment in a direction perpendicular to the 2D medical image in the 3D medical image to obtain at least one section in the 3D medical image; and
extracting image information on the section.

11. The method according to claim 9, wherein
at least one of the distance from the line segment to the center of the vertebral body and the angle of the line segment relative to the vertebral body is adjustable.

12. The method according to claim 1, wherein
the 2D medical image is a 2D ultrasound medical image,
the 3D medical image is a 3D ultrasound medical image, and
the 3D medical image is generated based on data acquired by an electronic scanning ultrasound probe or a mechanical scanning ultrasound probe.

13. A medical apparatus, characterized by comprising:
a memory storing instructions;
a processor configured to execute the instructions to:
identify a target object from a two-dimensional (2D) medical image to obtain information of the target object;
set a target position in the 2D medical image according to the information of the target object;
extract information corresponding to the target position from a three-dimensional (3D) medical image associated with the 2D medical image; and
generate a target medical image based on the extracted information.

14. A non-transitory computer-readable storage medium for storing a computer program, wherein when executed by a computer, the computer program causes the computer to:
identify a target object from a two-dimensional (2D) medical image to obtain information of the target object;
set a target position in the 2D medical image according to the information of the target object;
extract information corresponding to the target position from a three-dimensional (3D) medical image associated with the 2D medical image; and
generate a target medical image based on the extracted information.
